(19) United States
(12) Patent Application Publication
(10) Pub. No.: US 2023/0285430 A1
(43) Pub. Date: Sep. 14, 2023

(54) SPIROINDOLONE COMPOSITIONS AND METHODS FOR THE TREATMENT OF MALARIA

(71) Applicant: Novartis AG, Basel (CH)

(72) Inventors: Priyanga Wickramanayake, Basel (CH); Xueyan Wang, Basel (CH); Henricus Lambertus Gerrardus Maria Tiemessen, Basel (CH); Paolo De Marco, Basel (CH); Akouavi Kéli Norbette Asso, Basel (CH)

(21) Appl. No.: 18/020,995

(22) PCT Filed: Aug. 16, 2021
(86) PCT No.: PCT/IB2021/057531

§ 371 Date: Feb. 13, 2023

Related U.S. Application Data
(60) Provisional application No. 63/120,022, filed on Dec. 1, 2020.
(60) Provisional application No. 63/066,116, filed on Aug. 14, 2020.

(51) Int. Cl. A61K 31/675; A61K 9/107; A61K 47/42; A61K 47/24; A61K 45/06; A61P 33/06

(52) U.S. Cl. CPC A61K 31/675; A61K 9/1075; A61K 47/42; A61K 47/24; A61K 45/06; A61P 33/06

(57) ABSTRACT

The present invention is directed to compositions containing spiroindolone compounds, in particular, (1′R,3′S)-5,7′-dichloro-6′-fluoro-3′-methyl-2′,3′,4′,9′-tetrahydrospiro[indoline-3,1′-pyrido[3,4-b]indol]-2-one, and methods of administering in the treatment of malaria, in particular severe malaria

### FIELD

The present invention is directed to compositions and methods of administering spiroindolone compounds, in particular, (1′R,3′S)-5,7′-dichloro-6′-fluoro-3′-methyl-2′,3′,4′,9′-tetrahydrospiro[indoline-3,1′-pyrido[3,4-b]indol]-2-one, alone or in combination with other anti-malaria agents and therapies in the treatment of malaria and so relates to the fields of medicine, pharmacology, chemistry, and biology.

### BACKGROUND

Malaria, is an infectious disease caused by any of four protozoan parasites, Plasmodium falciparum, Plasmodium vivax, Plasmodium malariae and Plasmodium ovale. These four parasites are typically transmitted by the bite of an infected female Anopheles mosquito. Globally, billions people are at risk of malaria with an over 200 million cases and 500,000 deaths each year, mostly children under 5 years of age. 90% of malaria cases and 91% of malaria deaths occur in Africa, the vast majority in young children. The majority of deaths occur after infection with Plasmodium falciparum. Malaria is an acute febrile illness. In an individual, who is not immune to malaria, symptoms usually appear 10-15 days after the infective mosquito bite. The first symptoms may be mild and difficult to recognize as malaria. They arise because of the blood stage parasitemia with consequent erythrocyte rupture that produces the acute symptoms (spiking fever, rigors, malaise, headache and muscle aches), vascular compromise and organ involvement that can result in complications if not rapidly treated. P. falciparum malaria can progress to severe illness and death. Children with severe malaria frequently develop one or more of the following symptoms: severe anaemia, respiratory distress in relation to metabolic acidosis, or cerebral malaria. Also, in adults, multi-organ involvement is frequent. Thus, it is vital to initiate effective treatment shortly after diagnosis. The current World Health Organization (WHO) recommended treatment of severe malaria is artesunate administered either intravenously (iv) or intramuscularly (im) or quinine administered either iv or im. There are problems with both of these treatments. There are increasing reports of parasite resistance to artemisinins such as artesunate.

Additionally preparation and administration of artesunate injection is complicated. Quinine has to be given by slow infusion because of the risk of cardiovascular side effects. In addition, quinine has lower efficacy than artesunate.

Certain agents are now in the clinic for treating malaria. See for example, U.S. Pat. No. 8,053,422, which is incorporated herein by reference, which discloses the compound (1′R,3′S)-5,7′-dichloro-6′-fluoro-3′-methyl-2′,3′,4′,9′-tetrahydrospiro[indoline-3,1′-pyrido[3,4-b]indol]-2-one, (KAE609) and its preparation. In severe malaria, reduction of parasite count is one of the top treatment goals and therefore achieving the effective concentration in the systemic circulation is important. In addition, many of the severe malaria patients will not be able tolerate or take oral treatment. Therefore, intravenous (i.v.) administration is considered most suitable route of administration for KAE609 in severe malaria. However, KAE609 has limited solubility and in order to enable a safe and effective injectable (i.v., intramuscular (i.m.), intraparenteral (i.p.) etc.) administration and to elicit the required therapeutic effects, it needs to be solubilized. There are different ways to solubilize poorly soluble compounds for injectable administration. Some approaches involve the optimization of the pH, the use of co-solvents, surfactants, cyclodextrins, or parenteral fat emulsions. However, some of these approaches are not effective solubilizers for all compounds and they may be associated with adverse effects. Another possibility to solubilize poorly soluble compounds is the use of phospholipids (van Hoogevest P., Xiangli L., and Alfred F. “Drug delivery strategies for poorly water-soluble drugs: the industrial perspective” Expert Opinion on Drug Delivery 2011, 8(11), 1481-1500). However, the solubilization of a certain poorly soluble compound by phospholipids cannot be predicted. There remains a need for compositions and methods for treating malaria, particularly severe malaria. The aim of the current invention is to provide a composition, which can conveniently be used to solubilize and parenterally deliver KAE609 in a safe and efficacious manner. The present invention meets these needs by providing compositions and methods related to a KAE609 as summarized below.

### SUMMARY OF THE INVENTION

In one aspect, the present invention relates to compositions, and more specifically pharmaceutically acceptable formulations comprising the spiroindolone compound, (1′R,3′S)-5,7′-dichloro-6′-fluoro-3′-methyl-2′,3′,4′,9′-tetrahydrospiro[indoline-3,1′-pyrido[3,4-b]indol]-2-one (known as KAE609) or a pharmaceutically acceptable salt or solvate thereof, suitable for intravenous, intramuscular or intraparental administration to a subject in need thereof. The structure of KAE609 is shown below:

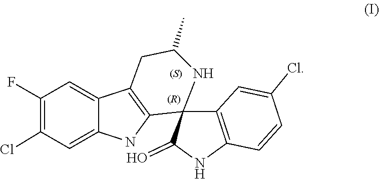

In one embodiment, the pharmaceutically acceptable formulation comprises KAE609, a first surfactant or micelle forming agent; and a second surfactant or micelle forming agent, wherein the composition comprises mixed micelles comprising the first and second surfactant or micelle forming agents. In various embodiments, the composition or pharmaceutically acceptable formulation comprises KAE609, and the first surfactant or micelle forming agent is a phospholipid, and more specifically the phospholipid is dimyristoyl glycero phosphatidyl choline (DMPC) or a lecithin such as Lecithin 100 or Lipoid S100 and the second micelle forming agent is a bile salt, such as sodium glycocholate. The formulation may be diluted (e.g., in an aqueous excipient) prior to administration to a patient. The invention provides a pharmaceutical, which enables a fast release of KAE609 after intravenous, intramuscular or intraparental administration.

### DETAILED DESCRIPTION OF THE INVENTION

This detailed description of the aspects and embodiments of the present invention is organized into sections as follows. Section I provides definitions of terms used herein. Section II describes the pharmaceutically acceptable formulations of the present invention. Section III provides treatment methods of the present invention. Section IV provides exemplary pharmaceutically acceptable formulations and treatment methods of the present methods. This detailed description is organized into sections only for the convenience of the reader, and disclosure found in any section is applicable to disclosure elsewhere in the specification.

### I. Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

“About” refers to greater than or less than 20% of a quantity and includes, but is not limited to greater than or less than 15%, greater than or less than 10%, and greater than or less than 5% of the quantity.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

The term “treat”, “treating”, “treated” or “treatment” includes the prevention, diminishment or alleviation of at least one symptom associated with or caused by the state, disease or disorder being treated.

The term “prevent”, “preventing” or “prevention” includes the prevention of at least one symptom associated with or caused by the state, disease or disorder being prevented.

The term “patient” includes organisms that are capable of suffering from, or afflicted or infected with, a parasitic disease, e.g. mammals such as humans, cows, horses, pigs, sheep, cats, dogs, goats, mice, rabbits, rats and transgenic non-human animals. In some embodiments the patient is a human, e.g. a human capable of suffering from, or afflicted with, malaria.

A “parasitic disease” includes disorders and states that are associated with a parasitic infection in a subject.

The term “pharmaceutical composition” or “pharmaceutical formulation” includes preparations, e.g. medicaments, suitable for administration to mammals, e.g. humans. “Pharmaceutically acceptable carrier, excipient, or diluent” refers to a carrier, excipient, or diluent that is useful in preparing a pharmaceutical composition that is generally safe, non-toxic and neither biologically nor otherwise undesirable, and includes a carrier, excipient, or diluent that is acceptable for human pharmaceutical use as well as veterinary use. A “pharmaceutically acceptable carrier, excipient, or diluent” includes both one and more than one such carrier, excipient, or diluent.

“Pharmaceutically acceptable salt” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts. The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Reduction” of a symptom or symptoms (and grammatical equivalents of this phrase) refers to decreasing the severity or frequency of the symptom(s), or elimination of the symptom(s).

“Therapeutically effective amount” or “effective amount” of a drug refers to an amount of a drug that, when administered to a patient with malaria, will have the intended therapeutic effect, e.g., alleviation, amelioration, palliation or elimination of one or more manifestations of malaria disease in the patient. A therapeutic effect does not necessarily occur by administration of one dose, and may occur only after administration of a series of doses. Thus, a therapeutically effective amount may be administered in one or more administrations.

### II. Pharmaceutical Formulations

In one aspect, the present invention relates to pharmaceutically acceptable formulations comprising the compound known as KAE609, or a pharmaceutically acceptable salt thereof, with a pharmaceutically acceptable carrier, excipient, or diluent. KAE609 is described in U.S. Pat. No. 8,053,422, incorporated herein by reference. In one embodiment, the pharmaceutically acceptable formulation is a solution comprising KAE609 and a first and second surfactant. Such a stable formulation of KAE609 is described in Example 1 and 2 below.

The structure of KAE609 is shown below:

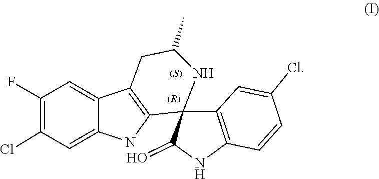

In other embodiments the present invention provides pharmaceutically acceptable formulations comprising other compounds described in U.S. Pat. No. 8,053,422.

In another aspect, the present invention provides a unit dose of a pharmaceutically acceptable formulation of KAE609. In one embodiment, the unit dose of the pharmaceutically acceptable formulation comprises about 10 mg to about 40 mg, including about 15 mg, 20 mg, 25 mg, 30 mg, or 35 mg, KAE609. A suitable unit dose pharmaceutically acceptable formulations of KAE609 are described in Example 1 below.

### III. Treatment Methods

In another aspect, the invention provides a method of treating malaria, and more specifically severe malaria, said method by administering a therapeutically effective amount of KAE609 in the range of about 10 mg to about 120 mg to a patient in need of such treatment. In another embodiment, KAE609 can be administered to human patients in a therapeutically effective amount in the range of about 10.5 mg, about 20 mg, about 30 mg, about 40 mg, about 75 mg, and up to about 120 mg. A human clinical trial protocol is described in Example 2 below. In one embodiment, KAE609 is administered by i.v. administration. In one embodiment, the therapeutically effective amount of KAE609 is administered at a frequency of at least once per day. In one embodiment, KAE609 is administered for a period of at least 1 day, at least 2 days, at least 3 days, at least 4 days up to at least 5 days. In other embodiments, longer periods of administration are employed. Various frequencies and periods of KAE609 administration for effective malaria treatment according to the present methods are described, for example, in Examples 3 and 4 below.

In one embodiment, the malaria treated is severe malaria.

In one embodiment, KAE609 may be administered in combination with another anti malaria agent.

In one embodiment, the present invention provides methods for treating malaria administering pharmaceutically acceptable formulations of the present invention comprising a spiroindolone compound other than KAE609, including those described in U.S. Pat. No. 8,053,422.

The invention, having been described in summary and in detail, is illustrated but not limited by the Examples below that provide pharmaceutically acceptable formulation of KAE609 of the present invention and demonstrate the efficacy of KAE609 administration to treat malaria in accordance with the present methods.

### EXAMPLES

The following examples are intended for illustration only and should not be construed to limit the scope of the invention. Example 1 describes formulations of the invention. Example 2 describes KAE609 monotherapy.

### Example 1: Pharmaceutical Formulations of KAE609

This example describes pharmaceutical formulations of KAE609 as well as the results of experimentation demonstrating the advantages of certain formulations. As discussed below, a formulation containing KAE609 and a first and second surfactant provide advantages over other formulations including higher solubility of KAE609, allowing for a more concentrated solution, greater stability on storage, and the absence of precipitation when the concentrated formulation is diluted into water, D5W or saline.

### Example 2: Pharmaceutical Formulations of KAE609

This example describes pharmaceutical formulations of KAE609.

TABLE 1
KAE609 compositions for formulation in 2 mL glass vials

| | | Composition | Composition |
|---|---|---|---|
| Component | | 1 (per mL | 2 (per mL |
| Description | Functionality | (mg)) | (mg)) |
| KAE609 | Drug | 15.0 | 10.0 |
| | substance | | |
| Soy Lecithin | Phospholipid | 102.6 | 0.0 |
| (phosphatidyl | (Micelle | | |
| choline | forming | | |
| isolated from | agent) | | |
| soy beans | | | |
| such as | | | |
| Lecithin 100 | | | |
| DMPC | Phospholipid | 0.0 | 67.0 |
| | (Micelle | | |
| | forming | | |
| | agent) | | |
| Sodium | Bile salt | 77.4 | 51.0 |
| Glycocholate | (Micelle | | |
| | forming | | |
| | agent) | | |
| Glycerol or | Isotonicity | 12.0 | 14.0 |
| Glycerin rein | agent | | |
| L-Histidine | Buffering | 0.78 | 0.78 |
| | agent | | |
| Disodium | Antioxidant | 0.5 | 0.1 |
| Edetate | | | |
| Dihydrate | | | |
| Hydrochloric | pH adjusting | as required | as required |
| acid | agent | to pH 6.5 | to pH 6.5 |
| Sodium | pH adjusting | as required | as required |
| hydroxide | agent | to pH 6.5 | to pH 6.5 |
| Water for | Solvent | Qs to 1.000 | Qs to 1.0 |
| injection | | mL up to | mL up to |
| | | 1028 mg | 1019 mg |
| Nitrogen | | q.s. | q.s. |
| 99.99% | | | |

### Example 3: Treatment of Severe Malaria in Human Patients Using KAE609 Monotherapy

A Phase 1/2, randomized, subject and investigator-blinded, placebo-controlled, single and multiple ascending iv dose study in healthy subjects design is conducted to demonstrate the safety and efficacy of KAE609 in accordance with the present invention. The study consists of two parts. In Part A sequential cohorts of 8 healthy subjects per cohort are randomized to receive successively higher single iv doses of KAE609 or matching placebo. In each cohort, subjects are randomized in a 3:1 ratio to KAE609 (6 subjects/cohort) or matching placebo (subjects/cohort). Four or more dose cohorts will be included with dose levels of from about 10.5 mg, 30 mg, 75 mg, and up to 120 mg.

In Part B sequential cohorts of 9 healthy subjects per cohort will be randomized to receive successively higher iv doses of KAE609 or matching placebo. Two planned dose cohorts will be included in Part B (B1 and B2). For Cohort B1, healthy subjects will receive a 60 mg dose of KAE609 or matching placebo iv q24h for 5 days. For Cohort B2, healthy subjects will start with a single loading dose of 120 mg or matching placebo on Day 1, followed by a maintenance dose of 75 mg or matching placebo q24h for the subsequent 4 days. In each cohort, subjects will be randomized in a 2:1 ratio to KAE609 (6 subjects/cohort) or matching placebo (3 subjects/cohort). For both parts of the study, each subject will participate in an up to 28-day screening period during which a full physical examination, medical history, drug screening, vital signs, ECG evaluation and safety laboratory tests evaluation will be performed. A review of safety and PK data is conducted at the completion of each cohort prior to dosing of the next subsequent cohort. Safety assessments may include physical examinations, ECGs, vital signs, standard clinical laboratory evaluations (hematology, blood chemistry, urinalysis) adverse event and serious adverse event monitoring.

### Example 4: Treatment of Severe Malaria in Human Patients Using KAE609 Monotherapy

A Phase 2 adaptive, randomized, active-controlled, open-label, sequential cohort, multicenter study to evaluate the efficacy, safety, tolerability and pharmacokinetics of intravenous cipargamin (KAE609) in participants aged ≥12 years old (in Cohorts 1-2) and <12 years old to ≥6 months (in Cohorts 3-5) with severe Plasmodium falciparum malaria in accordance with the present invention. The study consists of two parts. In Cohorts 1-2, participants will get randomized to one of the three treatment arms (2 dose of intravenous cipargamin (20 or 40 mg) or intravenous artesunate−comparator (2.4 mg/kg (for participants weighing at least 20 kg); 3 mg/kg (for participants weighing less than 20 kg)). After Cohort 2, interim analysis will be performed to take one best dose of intravenous cipargamin forward for Cohorts 3-5. Participants in Cohorts 3-5 with be randomized to one of two treatments arms ( intravenous cipargamin or intravenous artesunate−comparator). Standard of care (Coartem) will be given to all participants for 3 days as part of treatment.

For both parts of the study, each subject will participate in a screening period during which a full physical examination, medical history, drug screening, vital signs, ECG evaluation and safety laboratory tests evaluation will be performed. A review of safety and PK data is conducted at the completion of each cohort prior to dosing of the next subsequent cohort. Safety assessments may include physical examinations, ECGs, vital signs, standard clinical laboratory evaluations (hematology, blood chemistry, urinalysis) adverse event and serious adverse event monitoring.

While the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

1. A composition comprising N,N′-bis(2-bromoethyl)phosphorodiamidic acid (1-methyl-2-nitro-1H-imidazol-5-yl)methyl ester (KAE609) or a pharmaceutically acceptable salt thereof, a first surfactant or micelle forming agent; and a second surfactant or micelle forming agent, wherein the composition comprises mixed micelles comprising the first and second surfactant or micelle forming agents.

2. A composition of claim 1, wherein the first surfactant or micelle forming agent is selected from a phospholipid; and the second surfactant or micelle forming agent is selected from a bile salt.

3. A composition of claim 1, wherein further comprising at least one of an isotonicity agent, a buffering agent, and antioxidant, a pH adjusting agent and a solvent.

4. A composition of claim 1, wherein the phospholipid is selected from lecithin or dimyristoyl glycero phosphatidyl choline.

5. A pharmaceutically acceptable formulation suitable for intravenous, intramuscular or intraparental administration comprising a composition of claim 1.

6. A pharmaceutically acceptable formulation of claim 2 comprising N,N′-bis(2-bromoethyl)phosphorodiamidic acid (1-methyl-2-nitro-1H-imidazol-5-yl)methyl ester (KAE609) or a pharmaceutically acceptable salt thereof, in an amount of about 10 mg/ml to about 40 mg/ml.

7. A unit dose of the pharmaceutically acceptable formulation of any one of the preceding claims.

8. A method of treating malaria, said method comprising administering a composition or formulation of any of the preceding claims to a human patient in need of such treatment.

9. The method of claim 8, said method comprising administering a composition claim 1 or a formulation of claim 2 intravenously, intramuscularly or intraparentally.

10. The method of claim 8 or 9, wherein said malaria is severe malaria.

11. The method of any of the preceding claims, said method comprising administering a composition or pharmaceutically acceptable formulation of any of the preceding claims in an amount in the range of at least about 10 mg per day.

12. The method of any of the preceding claims, said method comprising administering a composition or pharmaceutically acceptable formulation of any of the preceding claims in an amount in the range of at least about 10 mg to about 120 mg per day.

13. The method of any of the preceding claims, wherein the KAE609 is administered at a frequency of at least once per day.

14. The method of any of the preceding claims, wherein the KAE609 is administered daily for at least 5 days.

15. The method of any of the preceding claims, wherein the malaria is sever malaria.

16. The method of any of the preceding claims, wherein KAE609 is administered in combination with another antimalarial agent.
